(19) United States
(12) Patent Application Publication
(10) Pub. No.: US 2021/0348097 A1
(43) Pub. Date: Nov. 11, 2021

(54) CELL CULTURE CHIP

(71) Applicant: Panasonic Intellectual Property Management Co., Ltd., Osaka (JP)

(72) Inventors: TAICHI NAKAMURA, Osaka (JP); NORIHITO TSUKAHARA, Kyoto (JP); KOUJI IKEDA, Hyogo (JP); KIYOTAKA TSUJI, Osaka (JP)

(21) Appl. No.: 17/231,070

(22) Filed: Apr. 15, 2021

(30) Foreign Application Priority Data
May 8, 2020 (JP) 2020-082693

(51) Int. Cl. C12M 3/06; C12M 1/12

(52) U.S. Cl. CPC C12M 23/16; C12M 21/08; C12M 25/14

(57) ABSTRACT

A cell culture chip has a stack structure formed by sequentially stacking: a first electrode provided on a main surface of a first board; a first partition wall layer including a first main flow path, and a first inlet flow path and a first outlet flow path connected to the first main flow path; a planar mesh structure sheet used as a scaffolding material for cells; a second partition wall layer including a second main flow path, and a second inlet flow path and a second outlet flow path connected to the second main flow path; and a second electrode provided on a main surface of a second board, in which the planar mesh structure sheet is sandwiched between the first partition wall layer and the second partition wall layer, and, among aperture ratios of a surface of the planar mesh structure sheet facing the first partition wall layer, an aperture ratio of a portion facing the first main flow path is greater than an aperture ratios of portions facing the first inlet flow path and the first outlet flow path, and, among aperture ratios of a surface of the planar mesh structure sheet facing the second partition wall layer, an aperture ratio of a portion facing the second main flow path is greater than an aperture ratios of portions facing the second inlet flow path and the second outlet flow path.

### BACKGROUND

### 1. Technical Field

The present disclosure relates to a cell culture chip.

### 2. Description of the Related Art

Recently, an organ on a chip (OoC) has been actively developed as a cell culture chip. (Refer to, for example, Japanese Patent Application No. 2019-506861 and Japanese Patent Unexamined Publication No. 2019-180354). The OoC is a cell culture chip that reproduces tissue functions in an organ on a microscale by culturing cells in an artificial micro-space in which glass, resin, and the like are combined, as illustrated in FIG. 39 of Japanese Patent Application No. 2019-506861.

By adding drug to cells cultured by using the cell culture chip, tests of the related art such as drug efficacy, toxicity test or absorption, metabolism, excretion, and the like of the drug, which are evaluated by animal tests using mice, can be performed in an artificial chip other than an organ.

### SUMMARY

A cell culture chip according to one aspect of the present disclosure has a stack structure formed by sequentially stacking a first board provided with a first electrode on a main surface, a first partition wall layer including a first main flow path, and a first inlet flow path and a first outlet flow path connected to the first main flow path, a planar mesh structure sheet used as a scaffolding material for cells, a second partition wall layer including a second main flow path, and a second inlet flow path and a second outlet flow path connected to the second main flow path, and a second board provided with a second electrode on a main surface, in which the planar mesh structure sheet is sandwiched between the first partition wall layer and the second partition wall layer, among aperture ratios of a surface of the planar mesh structure sheet facing the first partition wall layer, an aperture ratio of a portion facing the first main flow path is greater than an aperture ratios of portions facing the first inlet flow path and the first outlet flow path, and among aperture ratios of a surface of the planar mesh structure sheet facing the second partition wall layer, an aperture ratio of a portion facing the second main flow path is greater than an aperture ratios of portions facing the second inlet flow path and the second outlet flow path.

### BRIEF DESCRIPTION OF THE DRAWINGS

FIG. 1A is an exploded perspective view illustrating a member configuration of an example of a cell culture chip according to a first exemplary embodiment;

FIG. 1B is a top view of a scaffolding material which is one of members configuring the cell culture chip;

FIG. 1C is a cross-sectional view of the scaffolding material of FIG. 1B as viewed in an A-A direction;

FIG. 1D is a schematic transmission view illustrating a flow path in the central portion of the cell culture chip in which the scaffolding material of FIG. 1B is incorporated;

FIG. 2 is a flowchart of a method of manufacturing the scaffolding material of the cell culture chip according to the first exemplary embodiment;

FIG. 3 is a schematic cross-sectional view illustrating a cross-sectional structure of the cell culture chip of FIG. 1D as viewed in a B-B direction;

FIG. 4 is a schematic view illustrating a member configuration of an example of a cell culture chip of the related art;

FIG. 5 is a schematic cross-sectional view illustrating a cross-sectional structure of a central portion of an example of the cell culture chip of the related art;

FIG. 6A is a schematic transmission view illustrating a planar structure of a flow path of a central portion of an example of a task structure of the cell culture chip of the related art; and

FIG. 6B is a schematic cross-sectional view illustrating a cross-sectional structure as viewed in a D-D direction of FIG. 6A.

### DETAILED DESCRIPTION

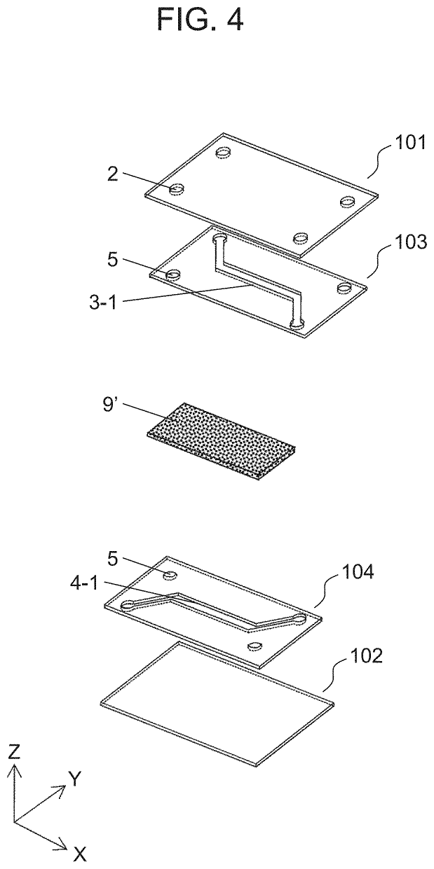

FIG. 4 is a schematic view illustrating a member configuration of an example of a general cell culture chip proposed by Japanese Patent Application No. 2019-506861. First, members configuring the cell culture chip of the related art will be described with reference to FIG. 4. In this cell culture chip, cells are cultured on a sheet of scaffolding material 9′ of the related art formed of a polymer material such as polyethylene phthalate or polystyrene. Scaffolding material 9′ of the related art is sandwiched between first partition wall layer 103 and second partition wall layer 104 in which first main flow path 3-1 and second main flow path 4-1 for supplying a liquid medium used for cell culture are formed respectively. Widths of first main flow path 3-1 and second main flow path 4-1 are generally formed within a range of 0.2 to 0.5 mm.

A sandwiching structure between first partition wall layer 103 and second partition wall layer 104 of scaffolding material 9′ is formed by a method of aligning and stacking respective configuration members of first partition wall layer 103, scaffolding material 9′, and second partition wall layer 104, interposing an adhesive therebetween, or the like. In addition, first partition wall layer 103 and second partition wall layer 104 are generally formed of silicone resin. The partition wall layers are including first main flow path 3-1 and second main flow path 4-1 that serve to be supplied with a medium from the outside of the chip and discharge the medium, and through-holes 5 that serve as alignment marks when first partition wall layer 103 is stacked on second partition wall layer 104.

First board 101 and second board 102 are stacked on external sides of first partition wall layer 103 and second partition wall layer 104, respectively. First board 101 and second board 102 serve as lids of first main flow path 3-1 and second main flow path 4-1 filled with a liquid medium. First board 101 and second board 102 are generally formed of glass having a thickness of approximately 0.3 to 1.0 mm, and first partition wall layer 103 and second partition wall layer 104 are stacked and bonded by a method of interposing an adhesive therebetween or the like. In addition, similar to first partition wall layer 103 or second partition wall layer 104, first board 101 is provided with through-holes 2 that serve as alignment marks when supplying and discharging a medium from the outside of the chip and stacking respective configuration members.

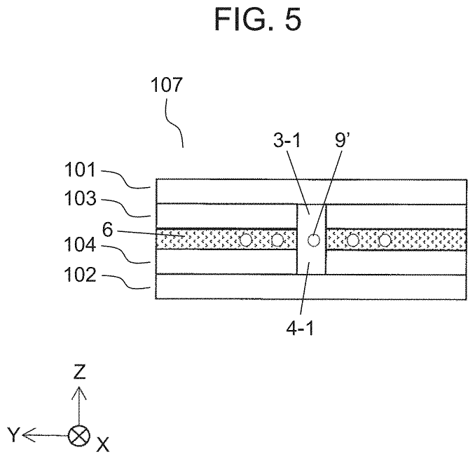

FIG. 5 is a schematic cross-sectional view illustrating a cross-sectional structure of a central portion of cell culture chip 107 after respective configuration members are stacked and bonded. A medium used for cell culture is divided into an upper portion and a lower portion via scaffolding material 9′ of the related art in which cells are cultured in first main flow path 3-1 of an upper flow path and in second main flow path 4-1 of a lower flow path.

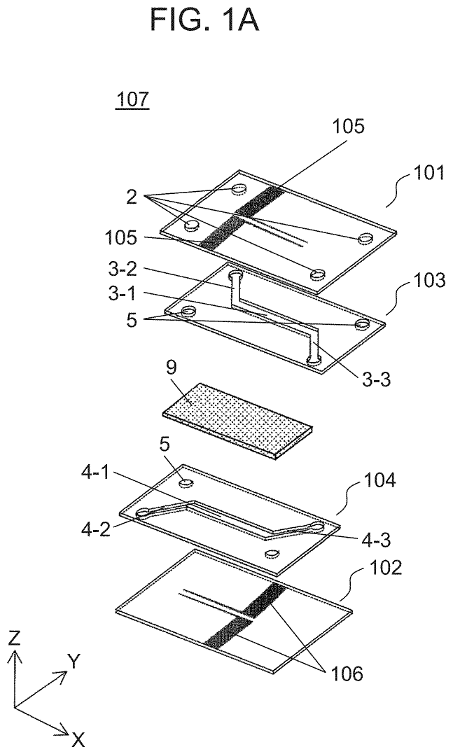

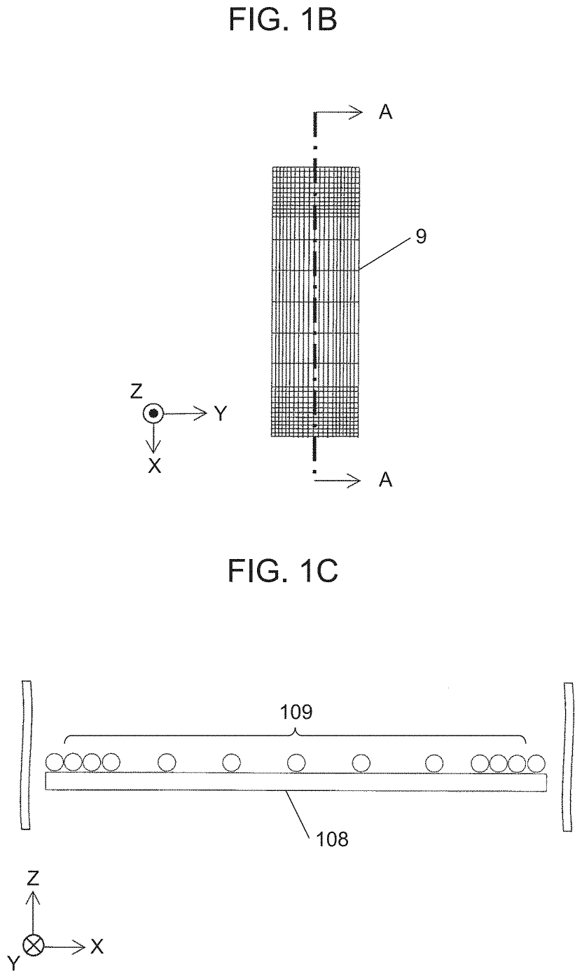

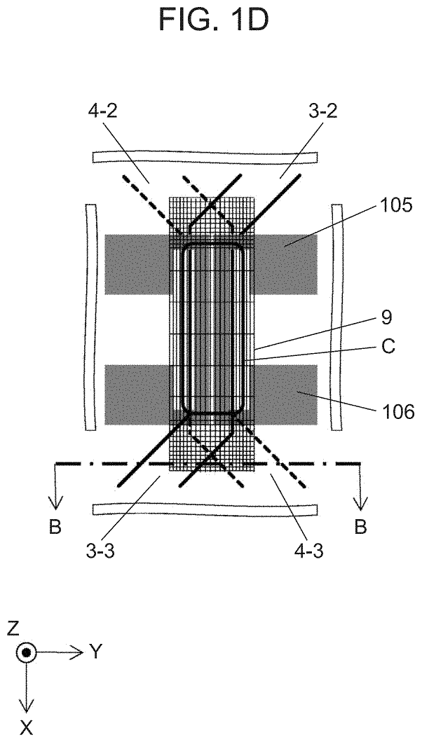

Due to this configuration, for example, when drug is added to the medium filling first main flow path 3-1 of first partition wall layer 103, the drug reaches a medium filling second main flow path 4-1 of second partition wall layer 104 through scaffolding material 9′ of the related art in which the cells are cultured. Thus, by analyzing medium component of second main flow path 4-1 of second partition wall layer 104 after a certain time, it is possible to evaluate drug efficacy, toxicity test or absorption, metabolism, and the like of the drug for the cells. Particularly, as illustrated in FIG. 1 of Japanese Patent Unexamined Publication No. 2019-180354, when a sheet having a planar mesh structure is used for scaffolding material 9′ of the related art, co-culture (for example, hepatocytes or intestinal cells, alternatively, cerebral blood barrier cells, cerebral nerve cells, or the like) of a cell sheet with a function closer to an organ is possible.

Further, as a thickness of the sheet having the planar mesh structure is reduced and an aperture ratio is increased, the cell sheets cultured in first main flow path 3-1 and second main flow path 4-1 are closer to each other. Therefore, since the function is closer to the organ, it is expected that accuracy of test results of drug efficacy, toxicity test or absorption, metabolism, and the like of the drug for the cells is increased.

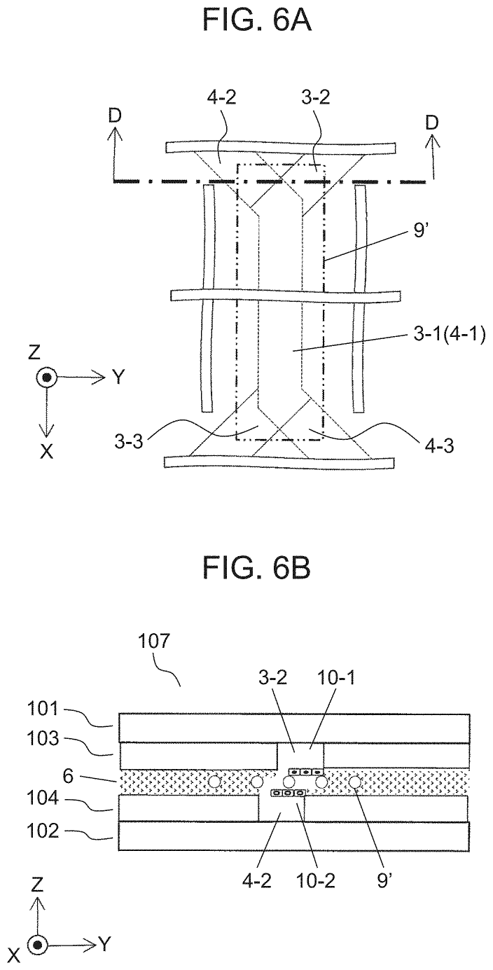

However, the general cell culture chip of the related art proposed by Japanese Patent Application No. 2019-506861 and the like has a problem that evaluation of cells performed by using the cell culture chip is inaccurate. Specifically, the present disclosure relates to improvement of a cell culture state and electrical evaluation. FIG. 6A is a schematic transmission view illustrating a planar structure of a flow path of a central portion of cell culture chip 107 of the related art to which the planar mesh structure sheet having a small thickness and a large aperture ratio is applied, as scaffolding material 9′ of the related art illustrated in FIG. 5. FIG. 6B is a schematic cross-sectional view illustrating a cross-sectional structure as viewed in a D-D direction of FIG. 6A. When a culture state of cells in the cell culture chip is electrically evaluated by a certain method, a space can be easily formed in cell sheets of upper cells 10-1 and lower cells 10-2, at an end portion where a mesh is easily enlarged during culturing, as illustrated in the schematic cross-sectional view of FIG. 6B. As a result, there is a problem that a short circuit (electrical conduction) occurs.

As described above, a problem to be solved by the present disclosure is to provide a cell culture chip that enables more accurate electrical evaluation while using a scaffolding material that enables co-culture of cell sheets in the cell culture chip.

A cell culture chip according to a first aspect has a stack structure formed by sequentially stacking a first electrode provided on a main surface of a first board, a first partition wall layer including a first main flow path, and a first inlet flow path and a first outlet flow path connected to the first main flow path, a planar mesh structure sheet used as a scaffolding material for cells, a second partition wall layer including a second main flow path, and a second inlet flow path and a second outlet flow path connected to the second main flow path, and a second electrode provided on a main surface of a second board, in which the planar mesh structure sheet is sandwiched between the first partition wall layer and the second partition wall layer, among aperture ratios of a surface of the planar mesh structure sheet facing the first partition wall layer, the aperture ratio of a portion facing the first main flow path is greater than the aperture ratios of portions facing the first inlet flow path and the first outlet flow path, and among aperture ratios of a surface of the planar mesh structure sheet facing the second partition wall layer, the aperture ratio of a portion facing the second main flow path is greater than the aperture ratios of portions facing the second inlet flow path and the second outlet flow path.

According to a cell culture chip according to a first aspect of the present disclosure, in s planar mesh structure sheet used as a scaffolding material, an aperture ratio of a portion facing a first main flow path is greater than aperture ratios of portions facing a first inlet flow path and a first outlet flow path, and an aperture ratio of a portion facing a second main flow path is greater than aperture ratios of portions facing a second inlet flow path and a second outlet flow path. Accordingly, it is possible to suppress occurrence of short circuits (electrical conductions) at a portion where the first inlet flow path and the second inlet flow path intersect and at a portion from which the first outlet flow path and the second outlet flow path branch off. Further, it is possible to provide a state in which cell sheets cultured in the first main flow path and the second main flow path are closer to each other. Therefore, in the cell culture chip, it is possible to increase accuracy of evaluation of cells and to perform more stable co-culture (for example, hepatocytes or intestinal cells, alternatively, cerebral blood barrier cells, cerebral nerve cells, or the like) of a cell sheet having a function closer to an organ. Thereby, since the function is closer to the organ, accuracy of test results of drug efficacy, toxicity test or absorption, metabolism, and the like of drug for cells is increased.

In a cell culture chip according to a second aspect, in the first aspect, a planar mesh structure sheet used as a scaffolding material is formed by crossing fibers of a polymer material at 30 degrees to 120 degrees, each fiber having a diameter of 1 μm to 50 μm.

In a cell culture chip according to a third aspect, in the first aspect or the second aspect, in the planar mesh structure sheet, an interval between fibers of a mesh structure of a scaffolding material corresponding to a main portion where an upper flow path and a lower flow path face each other and cells are cultured is 10 to 100 μm, and an interval between fibers of a mesh structure of a scaffolding material facing a portion to or from which the upper flow path and the lower flow path merge or branch off, is 1 to 10 μm.

Hereinafter, a cell culture chip according to an exemplary embodiment will be described with reference to the accompanying drawings.

In the drawings, substantially the same members are denoted by the same reference numerals.

### First Exemplary Embodiment

Hereinafter, cell culture chip 107 according to a first exemplary embodiment will be described. FIG. 1A is an exploded perspective view illustrating a member configuration of an example of cell culture chip 107 according to the first exemplary embodiment. FIG. 1B is a top view of scaffolding material 9 which is one of members configuring the cell culture chip. FIG. 1C is a cross-sectional view of the scaffolding material of FIG. 1B as viewed in an A-A direction. FIG. 1D is a schematic transmission view illustrating a flow path of a central portion of the cell culture chip in which scaffolding material 9 of FIG. 1B is incorporated. In the drawings, for the sake of convenience, a direction in which first main flow path 3-1 and second main flow path 4-1 extend is referred to as an X direction, a width direction perpendicular to the X direction in a plane is referred to as a Y direction, and a direction perpendicular to an XY plane is referred to as a Z direction.

Cell culture chip 107 has a stack structure in which first board 101, first partition wall layer 103, planar mesh structure sheet 9 used as a scaffolding material, second partition wall layer 104, and second board 102 are sequentially stacked. First board 101 is provided with first electrode 105 on a main surface thereof. First partition wall layer 103 includes first main flow path 3-1, and first inlet flow path 3-2 and first outlet flow path 3-3 connected to first main flow path 3-1. Second partition wall layer 104 includes second main flow path 4-1 and second inlet flow path 4-2, and second outlet flow path 4-3 connected to second main flow path 4-1. Second board 102 is provided with second electrode 106 on a main surface thereof. Planar mesh structure sheet 9 is sandwiched between first partition wall layer 103 and second partition wall layer 104. Among aperture ratios of a surface of planar mesh structure sheet 9 facing first partition wall layer 103, the aperture ratio of a portion facing first main flow path 3-1 is greater than the aperture ratios of portions facing first inlet flow path 3-2 and first outlet flow path 3-3. Among aperture ratios of a surface of planar mesh structure sheet 9 facing second partition wall layer 104, the aperture ratio of a portion facing second main flow path 4-1 is greater than the aperture ratios of portions facing second inlet flow path 4-2 and second outlet flow path 4-3. Accordingly, it is possible to suppress occurrence of short circuits (electrical conductions) at a portion where first inlet flow path 3-2 and second inlet flow path 4-2 intersect and at a portion from which first outlet flow path 3-3 and second outlet flow path 4-3 branch off. Further, it is possible to provide a state in which the cell sheets cultured in first main flow path 3-1 and second main flow path 4-1 are closer to each other. Therefore, in the cell culture chip, it is possible to increase accuracy of evaluation of cells and to perform more stable co-culture (for example, hepatocytes or intestinal cells, alternatively, cerebral blood barrier cells, cerebral nerve cells, or the like) of a cell sheet having a function closer to an organ. Thereby, since the function is closer to the organ, accuracy of test results of drug efficacy, toxicity test or absorption, metabolism, and the like of drug for cells is increased.

Hereinafter, respective members configuring cell culture chip 107 will be described in detail. Cell culture chip 107 is formed by sequentially aligning, stacking, and bonding respective configuration members of first board 101, first partition wall layer 103 having first main flow path 3-1, planar mesh structure sheet 9 used as a scaffolding material of cells, second partition wall layer 104 having second main flow path 4-1, and second board 102.

### First Board and Second Board

On a side of first board 101 facing first partition wall layer 103, first electrode 105 is formed of a wire, which has a width smaller than or equal to a width of first main flow path 3-1, along an internal side of first main flow path 3-1 and a wide wire drawn from the wire. Likewise, on a side of second board 102 facing second partition wall layer 104, second electrode 106 is formed of a wire, which has a width smaller than or equal to a width of second main flow path 4-1, along an internal side of second main flow path 4-1 and a wide wire drawn from the wire. By using first electrode 105 and second electrode 106, electrical resistance can be increased by forming a tight junction or the like between a cell sheet of upper cells 10-1 (not illustrated) to be cultured in first main flow path 3-1 and a cell sheet of lower cells 10-2 (not illustrated) to be cultured in second main flow path 4-1. As a result, culture states of the cell sheets can be stably evaluated.

Glass may be used for first board 101 and second board 102. Both first electrode 105 and second electrode 106 may be formed of indium tin oxide (ITO). Accordingly, since first board 101 and second board 102 and first electrode 105 and second electrode 106 are all transparent, cells to be cultured can be visually evaluated by using a microscope or the like. In addition to glass, a resin material such as polystyrene or acrylic may be used for first board 101 and second board 102, but it is preferable that first board 101 and second board 102 are transparent from the above viewpoint.

### First Partition Wall Layer and Second Partition Wall Layer

First partition wall layer 103 includes first main flow path 3-1, and first inlet flow path 3-2 and first outlet flow path 3-3 connected to first main flow path 3-1. Second partition wall layer 104 includes second main flow path 4-1 and second inlet flow path 4-2, and second outlet flow path 4-3 connected to second main flow path 4-1. In the present exemplary embodiment, polystyrene resin may be used for a molding material for forming first partition wall layer 103 having first main flow path 3-1 and second partition wall layer 104 having second main flow path 4-1. Adhesion of first board 101 and first partition wall layer 103 and adhesion of second board 102 and second partition wall layer 104 can be made by leaving the boards and partition wall layers in an atmosphere of 80° C. for approximately 2 hours after adhesive 6 is dispense-coated and the boards and partition wall layers are aligned and stacked. Alternatively, thermocompression bonding may be used as a method that does not use an adhesive. Here, when an adhesive is used, it is preferable to use a silicone-based material from a viewpoint of cytotoxicity.

### Planar Mesh Structure Sheet (Scaffolding Material)

Planar mesh structure sheet 9 used as a scaffolding material for cells is sandwiched between first partition wall layer 103 and second partition wall layer 104 in which first main flow path 3-1 formed in first partition wall layer 103 and second main flow path 4-1 formed in second partition wall layer 104 face each other and which are stacked so as to overlap each other. A method of sandwiching planar mesh structure sheet 9 used as a scaffolding material can be performed by dispense-coating first partition wall layer 103 and second partition wall layer 104 with an adhesive and aligning and stacking the partition wall layers, and then leaving the partition wall layers in an atmosphere of 80° C. for approximately 2 hours. Alternatively, thermocompression bonding may be used as a method that does not use an adhesive.

Planar mesh structure sheet 9 used as a scaffolding material is, for example, a planar mesh structure sheet formed of a fiber group of polystyrene. Further, planar mesh structure sheet 9 has, for example, a thickness of approximately 2 μm, an interfiber distance of approximately 3 μm in an outer circumferential portion thereof, and an interfiber distance of approximately 20 μm in a central portion thereof but is not limited thereto. Since a thickness corresponds to a diameter of a fiber, the thickness may be appropriately adjusted according to characteristics of the type of cells to be cultured but is preferably 1 μm to 50 μm. Further, interfiber distances corresponding to a portion where first inlet flow path 3-2 and second inlet flow path 4-2 intersect and a portion from which first outlet flow path 3-3 and second outlet flow path 4-3 branch off is preferably less than or equal to 10 μm from a viewpoint of suppressing occurrence of a short circuit (electrical conduction) even in a portion where upper cells 10-1 and lower cells 10-2 are difficult to be cultured in a sheet shape. Furthermore, cell sheets cultured in first main flow path 3-1 and second main flow path 4-1 may be closer to each other but an interfiber distance corresponding to first main flow path 3-1 and second main flow path 4-1 is preferably greater than or equal to 10 μm or less than or equal to 100 μm when considering an initial cell size to be cultured. Further, polystyrene with less concern about cytotoxicity described above can be used for resin forming scaffolding material 9, but the resin is not limited thereto as long as concern about cytotoxicity is less. Resin for forming scaffolding material 9 may be, for example, a polylactic acid type or a silicone type, but is preferably a polymer material because flexibility is required as a function as a scaffold of cells. In a cross-sectional view of planar mesh structure sheet 9 of FIG. 1C as viewed in an A-A direction, planar mesh structure sheet 9 which is a scaffolding material has a planar mesh structure configured by a two-layer structure of first layer spinning group 108 and second layer spinning group 109. First layer spinning group 108 and second layer spinning group 109 are bonded by being partially entwined, which will be described below.

Furthermore, a sparse and dense structure that is characteristics of planar mesh structure sheet 9 will be described in detail with reference to a schematic transmission view illustrating a flow path of a central portion of the cell culture chip of FIG. 1D. As illustrated in FIG. 1D, a portion where fibers of planar mesh structure sheet 9 which is the scaffolding material of FIG. 1C is dense, that is, a portion of a mesh with a small aperture ratio faces a portion to which first inlet flow path 3-2 and second inlet flow path 4-2 merge or a portion from which first outlet flow path 3-3 and second outlet flow path 4-3 branch off. For example, the portion of the mesh with the small aperture ratio is alternate long and short dash line B-B in FIG. 1D. Further, a portion where fibers are sparse, that is, a portion of a mesh with a large aperture ratio faces a main portion where cells are cultured in first main flow path 3-1 and second main flow path 4-1, for example, a C region in FIG. 1D.

Method of Manufacturing Planar Mesh Structure Sheet 9 which is Scaffolding Material

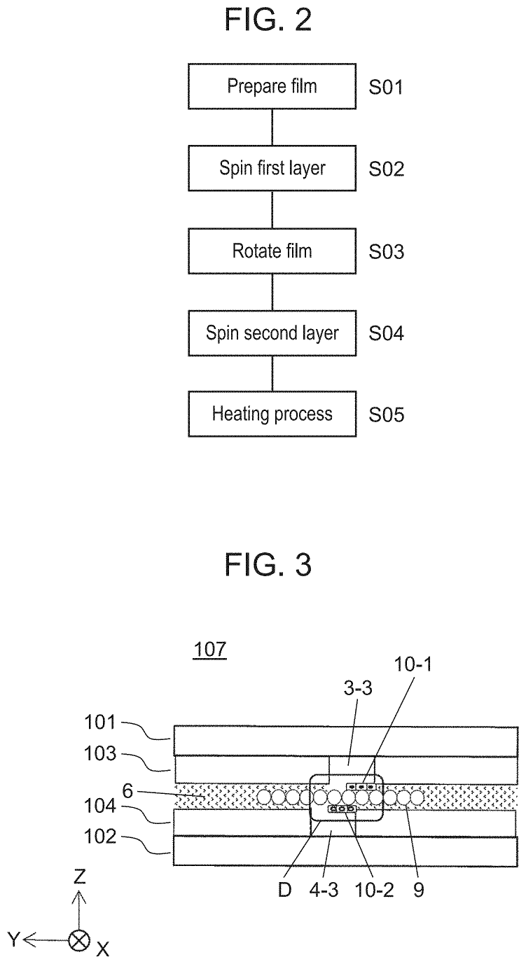

FIG. 2 is a flowchart of a method of manufacturing planar mesh structure sheet 9 which is a scaffolding material for forming the cell culture chip according to the first exemplary embodiment.

(1) S01 is a process of preparing a film. It is preferable that a film surface has appropriate peeling off in fluorine processing or the like. This is because an adhesive function to a fiber is required when spinning the fiber on a film in S02 and S04 to be described below, and in the first exemplary embodiment, the peeling off function from the film is required when incorporating scaffolding material 9 into the cell culture chip later.

(2) S02 is a process of spinning a first layer. A solution obtained by melting a polymer material used as scaffolding material 9 for the cell culture chip according to the first exemplary embodiment by heating or swelling with an organic solvent is applied onto a film prepared in S01 in a fine line shape in the same direction. Here, the polymer material supplied in a melting or solution state is naturally cooled or naturally dried to form a fiber only in a solid state. In the first exemplary embodiment, for example, polystyrene with low cytotoxicity is employed as the polymer material, and fibers, each having a diameter of approximately 2 μm, are coated, in the same direction at equal intervals of 3 μm, with a solution obtained by swelling pellet-shaped polystyrene in N,N-dimethylformamide (N-DMF) which is an organic solvent by 30% by weight.

(3) S03 is a process of rotating the film, which is obtained by spinning the first layer in S02, at a predetermined angle in the same surface. In the first exemplary embodiment, a mesh structure is formed in which the first layer spun in S01 and a second layer to be spun in S04 to be described below are orthogonal to each other by being rotated by 90 degrees. However, a crossing angle may be 30 degrees to 120 degrees, but it is preferable to cross at 90 degrees from a viewpoint of holding a mesh of a scaffolding material against an external force.

(4) S04 is a process of spinning the second layer on the film rotated by 90 degrees in S03. A solution obtained by melting a polymer material used as scaffolding material 9 by heating or swelling with an organic solvent is applied onto a film prepared in S03 in a fine line shape in the same direction. In the first exemplary embodiment, polystyrene with low cytotoxicity is employed as the polymer material in the same manner as in S02, and fibers, each having a diameter of approximately 2 μm, are coated, in the same direction, with a solution obtained by swelling pellet-shaped polystyrene in N,N-dimethylformamide (N-DMF) which is an organic solvent by 30% by weight. However, as illustrated in the cross-sectional view of FIG. 1C, a first half of the coating is at an interval of 3 μm, a middle stage is at an interval of 20 μm, and a second half is at an interval of 3 μm.

(5) S05 is a process of heating the fiber mesh on the film prepared up to process S04. Specifically, by heating the polymer material (polystyrene in the first exemplary embodiment) at a temperature higher than or equal to a glass transition point and lower than a melting point for a certain time, a majority portion of contact points between an upper portion of the first layer and a lower portion of the second layer is entwined. A planar mesh structure sheet used as a scaffolding material can be obtained based on the above.

Accordingly, a scaffolding material, which faces an outer circumferential portion of the cell culture chip, that is, a portion to which first inlet flow path 3-2 and second inlet flow path 4-2 merge or a portion from which first outlet flow path 3-3 and second outlet flow path 4-3 branch off, has a dense mesh structure and a small mesh opening, that is, an aperture ratio of a mesh is reduced. Meanwhile, a scaffolding material, which faces a central portion of the cell culture chip, that is, a main portion where cells are cultured in first main flow path 3-1 and second main flow path 4-1, has a sparse mesh structure and a large mesh opening, that is, an aperture ratio of a mesh is increased.

FIG. 3 is a schematic cross-sectional view illustrating a cross-sectional structure of the cell culture chip of FIG. 1D as viewed in a B-B direction. In FIG. 3, in a region D of an outer circumferential portion of the cell culture chip, a mesh is easily enlarged during culturing at a portion from which first outlet flow path 3-3 and second outlet flow path 4-3 branch off. Therefore, a space can be easily formed in cell sheets of upper cells 10-1 and lower cells 10-2, and cells are difficult to be cultured in a sheet shape. However, in the cell culture chip according to the first exemplary embodiment, by using scaffolding material 9, a portion having a dense mesh structure and a small mesh opening, that is, a portion having a small aperture ratio of a mesh faces the portion from which first outlet flow path 3-3 and second outlet flow path 4-3 branch off. Therefore, even when a mesh is slightly large during culturing, formation of a gap is prevented and a scaffolding material itself becomes electrical resistance, and thus, it is possible to suppress occurrence of a short circuit (electrical conduction). The same applies to a portion to which first inlet flow path 3-2 and second inlet flow path 4-2 merge.

Thereby, electrical resistance can be increased by forming a tight junction or the like between a cell sheet of upper cells 10-1 to be cultured in first main flow path 3-1 and a cell sheet of lower cells 10-2 to be cultured in second main flow path 4-1. As a result, it is possible to suppress occurrence of a short circuit, and to stably evaluate a culture state of a cell sheet.

Further, in the first exemplary embodiment, in order to make an aperture ratio of a planar mesh structure sheet greater in a first main flow path than in a first inlet flow path and a first outlet flow path and to make an aperture ratio of a planar mesh structure sheet greater in a second main flow path than in a second inlet flow path and a second outlet flow path, an interval between fibers is controlled by keeping a fiber diameter constant, but the present disclosure is not limited to this method. For example, a fiber diameter may be controlled by keeping an interval between fibers constant.

Specifically, for example, a fiber diameter of second layer spinning group 109 is set to 8 μm in a first half, 2 μm in a middle stage, and 8 μm in a second half. Accordingly, a scaffolding material, which faces a portion to which first inlet flow path 3-2 and second inlet flow path 4-2 merge, or a portion from which first outlet flow path 3-3 and second outlet flow path 4-3 branch off, has a dense mesh structure and a small mesh opening, that is, an aperture ratio of a mesh can be reduced. Meanwhile, a scaffolding material, which faces a main portion where cells are cultured in first main flow path 3-1 and second main flow path 4-1, has a sparse mesh structure and a large mesh opening, that is, an aperture ratio of a mesh can be increased.

The present disclosure includes appropriate combination of any exemplary embodiment and/or example among various exemplary embodiments and/or examples described above, and effects of the respective exemplary embodiments and/or examples can be obtained.

According to a cell culture chip of the present disclosure, in the cell culture chip, it is possible to increase accuracy of evaluation of cells and to perform more stable co-culture (for example, hepatocytes or intestinal cells, alternatively, cerebral blood barrier cells, cerebral nerve cells, or the like) of a cell sheet having a function closer to an organ. Thereby, quality of cell culture can be increased, and accuracy of test results of drug efficacy, toxicity test or absorption, metabolism, and the like of drug for cells is increased.

What is claimed is:

1. A cell culture chip having a stack structure formed by sequentially stacking:
a first electrode provided on a main surface of a first board;
a first partition wall layer including a first main flow path, and a first inlet flow path and a first outlet flow path connected to the first main flow path;
a planar mesh structure sheet used as a scaffolding material for cells;
a second partition wall layer including a second main flow path, and a second inlet flow path and a second outlet flow path connected to the second main flow path; and
a second electrode provided on a main surface of a second board,
wherein the planar mesh structure sheet is sandwiched between the first partition wall layer and the second partition wall layer,
among aperture ratios of a surface of the planar mesh structure sheet facing the first partition wall layer, an aperture ratio of a portion facing the first main flow path is greater than an aperture ratios of portions facing the first inlet flow path and the first outlet flow path, and
among aperture ratios of a surface of the planar mesh structure sheet facing the second partition wall layer, an aperture ratio of a portion facing the second main flow path is greater than an aperture ratios of portions facing the second inlet flow path and the second outlet flow path.

2. The cell culture chip of claim 1,
wherein the planar mesh structure sheet used as the scaffolding material is formed by crossing fibers of a polymer material at 30 degrees to 120 degrees, each fiber having a diameter of 1 μm to 50 μm.

3. The cell culture chip of claim 1,
wherein, in the planar mesh structure sheet, an interval between fibers of a mesh structure of a scaffolding material corresponding to a main portion where an upper flow path and a lower flow path face each other and cells are cultured is 10 to 100 μm, and an interval between fibers of a mesh structure of a scaffolding material facing a portion to or from which the upper flow path and the lower flow path merge or branch off, is 1 to 10 μm.
